(19) United States
(12) Patent Application Publication
(10) Pub. No.: US 2023/0000925 A1
(43) Pub. Date: Jan. 5, 2023

(54) METHOD OF USING ALLOGENEIC HAPLOIDENTICAL ADIPOSE TISSUE-DERIVED STROMAL VASCULAR FRACTION IN THE TREATMENT OF HUTCHINSON-GILFORD PROGERIA SYNDROME PATIENT

(71) Applicant: Jaewoo PAK, Seoul (KR)

(72) Inventor: Jaewoo PAK, Seoul (KR)

(21) Appl. No.: 17/903,121

(22) Filed: Sep. 6, 2022

Related U.S. Application Data
(62) Division of application No. 16/823,885, filed on Mar. 19, 2020.

(51) Int. Cl. A61K 35/35; A61K 9/00; A61K 38/48

(52) U.S. Cl. CPC A61K 35/35; A61K 9/0019; C12Y 304/24007; A61K 38/4886

(57) ABSTRACT

A method of treating a subject with Hutchinson-Gilford progeria syndrome includes administering to the subject a composition which includes an allogeneic haploidentical adipose tissue-derived stromal vascular fraction (SVF). The an allogeneic haploidentical adipose tissue-derived SVF may be produced by a process including centrifuging an allogeneic haploidentical adipose tissue lipoaspirate to obtain a packed adipose tissue, mixing the packed adipose tissue with collagenase, mincing the packed adipose tissue mixed with the collagenase by using a homogenizer, incubating the minced adipose tissue, centrifuging the incubated adipose tissue to separate and remove the collagenase, and repeating the centrifuging to obtain the stromal vascular fraction.

### CROSS REFERENCE TO RELATED APPLICATIONS AND CLAIM OF PRIORITY

This application is a divisional application of U.S. application Ser. No. 16/823,885, filed on Mar. 19, 2020, the entire contents of which are incorporated herein by reference.

### BACKGROUND

### 1. Field of the Invention

The present invention provides a method of using allogeneic haploidentical adipose tissue-derived stromal vascular fraction (adipose SVF) in the treatment of a Hutchinson-Gilford Progeria Syndrome patient. Specifically, it provides the method for the use of allogeneic haploidentical adipose tissue-derived SVF and its therapeutic effect.

### 2. Background Art

Hutchinson-Gilford Progeria Syndrome (HGPS), also known as Progeria, is a very rare, fatal genetic disorder in the LMNA gene, which encodes for two nuclear membrane proteins called, lamins A and C.

In normal conditions, LMNA gene produces prelamin A, which then undergoes a series of processing steps, including farnesylation of C terminus, a first cleavage and a second cleavage, as shown in FIG. 1A. In normal conditions, the farnesyl group of the C-terminus is removed off in the second cleavage of the processing, transforming prelamin A to lamin A (Harhouri, et al., Nucleus, 9, 246-257 (2018); Sinha, et al., Indian J. Med. Res., 139, 667-674 (2014)).

In HGPS patients, there is a mutation in LMNA gene, in which there is a deletion of genetic codes responsible for 50 amino acids at the C-terminus of prelamin A. However, in HGPS patients, there is a mutation in the LMNA gene, in which there is a deletion of genetic codes responsible for the 50 amino acids near the C terminus, disrupting the second cleavage site. With the disruption of the second cleavage site, the farnesyl group that supposed to be removed off from the C-terminus remains, as shown in FIG. 1B. The abnormal farnesylated prelamin A, which is also called progerin, accumulates and becomes incorporated into the nuclear membrane lamina and exerts damage to cells as HGPS patients age (Harhouri, et al., Nucleus, 9, 246-257 (2018); Sinha, et al., Indian J. Med. Res., 139, 667-674 (2014)). Due to the laminopathy created by progerin, HGPS patients show growth retardation early in their childhood along with other symptoms of physiological aging: loss of hair, skin thinning, joint rigidity, osteoporosis, and so on. The average height growth rate in HGPS patients is about 3.5 cm per year and the weight growth rate is about 0.5 kg per year during the ages of 1 to 10 (Merideth et al., N Engl. J. Med., 358, 592-604 (2008)). Their average age of survival is around 13.5 to 14.5 years and the life expectancy ranges 8 to 21, cardiovascular diseases being the major causes of death (Harhouri, et al., Nucleus, 9, 246-257 (2018); Sinha, et al., Indian J. Med. Res., 139, 667-674 (2014)).

Recently, lonafarnib, a farnesyltransferase inhibitor, which is also used as a chemotherapeutic agent, has been tried to treat the HGPS patients, resulting in the possibility of improving the mortality rate of the patients by 1.6 years or more (Gordon et al., JAMA, 319, 1687-1695 (2018); Gordon et al., Circulation, 130, 27-34 (2014)). Lonafarnib blocks the farnesylation process of prelamin A (Gordon et al., JAMA, 319, 1687-1695 (2018)). The end protein is modified progerin with shorter amino acids but without farnesyl group attached (FIG. 1C). In other words, the end product is a new product, identical to neither lamin A protein nor progerin (Gordon et al., JAMA, 319, 1687-1695 (2018)). Consequently, taking lonafarnib is not free of side effects. Accumulation of non-farnesylated prelamin A has caused cardiomyopathy in a mouse model (Davies, et al., Hum. Mol. Genet., 19, 2682-2694 (2010)). Therefore, the side effects of taking the medication had been the major drawback for the HGPS patient being presented in this case report.

A prelamin A polypeptide chain has its C-terminal -CaaX box (C: cysteine; aa: two aliphatic amino acids; and X: any amino acid). The α-helical rod domain is divided into segments for displaying the progerin defect. The first cleavage was carried out by the zinc metalloprotease (Zmpste24) or Ras-converting enzyme (RCE1) and the second cleavage by Zmpste24. FIGS. 1A to 1C were prepared using data adapted from Gordon et al. (Gordon et al., JAMA, 319, 1687-1695 (2018)).

Mesenchymal stem cells (MSCs) are defined as multipotent stem cells that (i) can adhere to plastics; (ii) can express the surface molecules such as CD105 and CD73, and lack expression of CD45, CD34, CD14 or CD11b, CD79a or CD19, and HLA-DR; and (iii) can differentiate to osteocytes, adipocytes, and chondrocytes (Dominici, et al., Cytotherapy, 8, 315-317 (2006)). In 2001 and 2002, Zuk et al. showed that adipose tissue in the form of a stromal vascular fraction (SVF) contains many multipotent stem cells that have the characteristics defined as MSCs (Zuk, et al., Mol. Biol. Cell., 13, 4279-4295 (2002)). MSCs, or adipose tissue-derived mesenchymal stromal cells, are known to produce a large amount of exosomes that are rich in active enzymes, proteins, and other cytoplasmic/nuclear constituents (Lai, et al., Humana Press, New York, N.Y., USA., 39-61 (2013); Lai, et al., Regen. Med., 8, 197-209 (2013); Arslan, et al., Stem Cell Res., 10, 301-312 (2013)). Adipose tissue-derived stem cells (ASCs) are one type of MSCs that can be obtained through manufacturing adipose SVF. This form of MSCs has been used in human patients since 2009 by injecting intra-articularly and intravenously without any serious side effects reported (Berman, et al., Am. J. Cosmetic Surg., 1-14 (2017); Pak, et al., BMC Musculoskelet. Disord., 14, 337 (2013)).

### SUMMARY

According to an aspect of the present invention, a composition for treating Hutchinson-Gilford progeria syndrome includes a stromal vascular fraction (SVF).

According to an aspect of the present invention, the stromal vascular fraction (SVF) may be produced by a process including centrifuging an allogeneic haploidentical adipose tissue lipoaspirate to obtain a packed adipose tissue, mixing the packed adipose tissue with collagenase, mincing the packed adipose tissue mixed with the collagenase by using a homogenizer, incubating the minced adipose tissue, centrifuging the incubated adipose tissue to separate and remove the collagenase, and repeating the centrifuging to obtain the stromal vascular fraction.

According to an aspect of the present invention, a method of treating a subject with Hutchinson-Gilford progeria syndrome includes preparing allogeneic haploidentical adipose tissue-derived stromal vascular fraction (SVF) from a donor without Hutchinson-Gilford progeria syndrome, and administering to the subject the allogeneic haploidentical adipose tissue-derived stromal vascular fraction.

According to an aspect of the present invention, the preparing of the allogeneic haploidentical adipose tissue-derived stromal vascular fraction includes obtaining a lipoaspirate from the donor, and isolating an adipose stromal vascular fraction (SVF) from the lipoaspirate to prepare the allogeneic haploidentical adipose tissue-derived stromal vascular fraction.

According to an aspect of the present invention, the isolation of the adipose stromal vascular fraction (SVF) from the lipoaspirate includes centrifuging the lipoaspirate to obtain a packed adipose tissue, mixing the packed adipose tissue with collagenase, mincing the packed adipose tissue by using a homogenizer, incubating the minced adipose tissue, centrifuging the incubated adipose tissue to separate and remove the collagenase, and repeating the centrifuging to obtain the stromal vascular fraction.

According to an aspect of the present invention, the administration includes intravenous push administration.

According to an aspect of the present invention, the administration includes a first intravenous push administration and a second intravenous push administration one week after the first intravenous push administration.

### BRIEF DESCRIPTION OF THE DRAWINGS

FIG. 1A shows posttranslational processing steps producing lamin A in a normal cell.

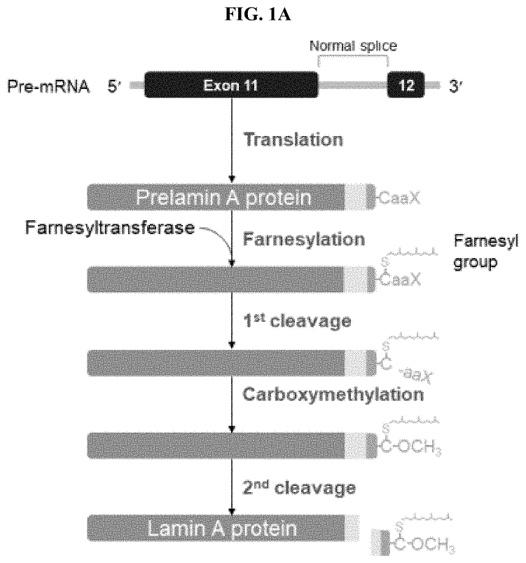

FIG. 1B shows posttranslational processing steps producing progerin in an HGPS cell.

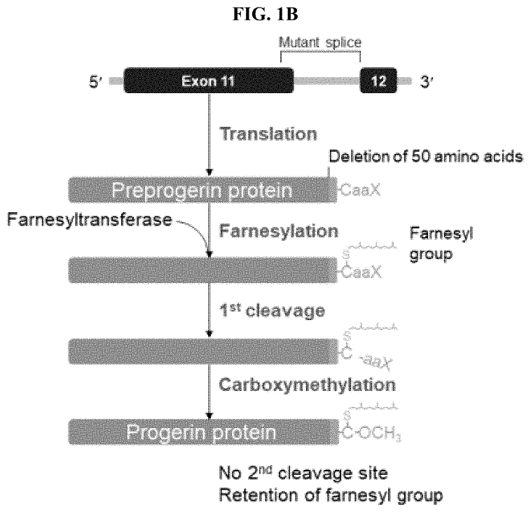

FIG. 1C shows posttranslational processing steps producing modified progerin in a lonafarnib-treated HGPS cell.

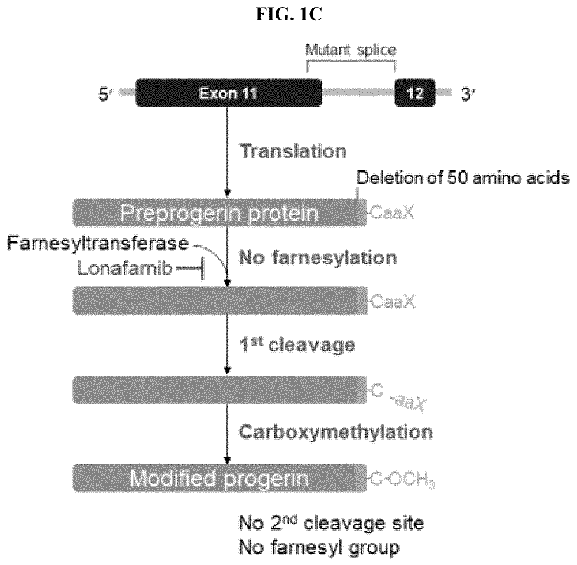

FIG. 2 shows outcomes obtained 12, 44, 62, and 134 days after the completion (the second adipose SVF injection) of treatment of the HGPS patient.

### DETAILED DESCRIPTION

According to an exemplary embodiment of the present invention, there is a provided a method of treating a subject with Hutchinson-Gilford progeria syndrome.

The method may include preparing allogeneic haploidentical adipose tissue-derived stromal vascular fraction (SVF) from a donor without Hutchinson-Gilford progeria syndrome, and administering to the subject the allogeneic haploidentical adipose tissue-derived stromal vascular fraction.

Extracellular vesicles (EVs), including exosomes released by MSCs, from healthy individuals may contain the correct form of prelamin A protein. Since MSCs from a healthy individual may contain all enzymes and proteins necessary to maintain normal homeostasis, it can be postulated that donor MSCs from a healthy individual may contain sufficient amount of correct form of prelamin A to be processed to Lamin A. That is, extracellular vesicles (EVs), including exosomes released by MSCs, from healthy individuals may contain the correct form of prelamin A protein. Thus, by transferring the normal prelamin A to the diseased target cells via EVs (e.g., exosomes) in HGPS patients, the target cells may be able to be replenished and be transformed into normal cells. We present a case of an HGPS patient who responded positively to injections of allogeneic haploidentical adipose tissue-derived SVFs containing MSCs by showing rapid height and weight growth along with increased blood level of insulin-like growth factor-1. Thus, an embodiment of the present invention provides a method for isolating and using allogeneic haploidentical adipose tissue-derived SVF, and a case of an HGPS patient who received allogeneic, haploidentical adipose tissue-derived SVF containing MSCs.

We developed methods of the liposuction and isolation of adipose SVF from the donor who was the patient's mother, and adipose SVF injection to treat a HGPS patient. The detailed description of the procedure describing the liposuction, the isolation of adipose SVF, and adipose SVF injection was described below in Example 1.

### Example 1

### Patient

The patient was a 12.8-year-old male with HGPS diagnosed in early childhood. He had previously tried lonafarnib for about 7 years from 2010 to 2017. He decided to discontinue the medication due to side effects of skin changes, chronic nausea which become very severe while traveling (patient temporarily quit the medication while traveling), and loss of energy described as a weakness. For about 2 years before visiting the Mipro Medical Clinic in July 2019, the patient was only on vitamins, including vitamins (liver vitamins containing riboflavin 5 mg, thiamine 10 mg, and ursodeoxycholic acid 50 mg) for liver and the chronic elevation of liver enzymes, and vitamin D.

This case report complies with the Declaration of Helsinki. Further, in 2009, the Korean Food and Drug Administration has allowed uses of adipose SVF (not the expanded and cultured ASCs) for medical treatments (Korean Food and Drug Administration (KFDA). Chapter 2, Section 14. (2009)). Informed consent was obtained from the patient's guardian, accordingly.

The inclusion criteria, exclusion criteria, and outcome endpoints were listed as follows:

Inclusion criteria: (i) diagnosis of HGPS; (ii) males or females; (iii) age over 10; and (iv) unwillingness to proceed with lonafarnib medication

Exclusion Criteria: (i) concomitant connective tissue disease thought to impact the HGPS (i.e. lupus, RA, and fibromyalgia); (ii) concomitant endocrine disorder that might impact results (i.e. hypothyroidism and diabetes); (iii) concomitant neurologic disorder that might impact results (i.e. peripheral neuropathy and multiple sclerosis); (iv) active cardiac disease; and (v) active pulmonary disease requiring medication usage.

Outcome endpoints (obtained 12, 44, 62, and 134 days after the completion of treatments): (i) pre- and post-treatment height; (ii) pre- and post-treatment weight; and (iii) pre- and post-treatment IGF-1 levels.

For two weeks before the procedure, the patient was restricted from taking any steroids and other herb medications.

### Liposuction, Isolation of Adipose SVF, and Adipose SVF Injection

After type and matching, the donor (in this case, the patient's mother) went through two separate occasions of liposuction, one week apart.

The patient was brought into an operating room and was placed in a supine position. The patient's abdominal area was cleaned with 5% betadine (povidone-iodine) and then draped using the sterile technique. Using 2 mL of 2% lidocaine without epinephrine with a needle (25gauge, 1½ inch) in 5-mL syringe, the site of incision to-be-made was anesthetized. Again, using 5 mL of tumescent solution (500 mL normal saline, 40 mL 2% lidocaine, 20 mL 0.5% marcaine, 0.5 mL epinephrine 1:1000) in 10-mL syringe with a needle (25gauge, 1½ inch), the site of incision to-be-made anesthetized. Using No 11 blade, incisions of approximately 0.5 cm below the umbilicus were made to the left and right side of the abdomen. Then, the whole lower abdomen area was anesthetized using 700 to 800 mL of the tumescent solution (500 mL normal saline, 40 mL 2% lidocaine, 20 mL 0.5% marcaine, 0.5 mL epinephrine 1:1000). Using a 3.0 mm cannula connected to 60-mL Luer-Lock syringe, 100 mL of adipose tissue in the form of lipoaspirates was obtained, and the tumescent solution was separated by gravity.

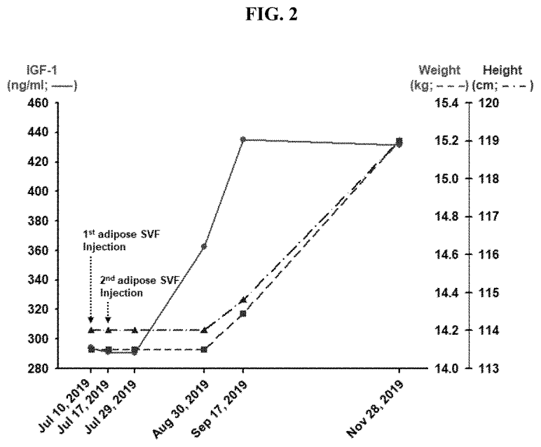

The 100 mL of lipoaspirates were divided equally and transferred into two 60-mL centrifuge syringe barrels of Tissue Centrifuge Kit (TCK) developed by us and approved by the FDA (CDRH). Afterward, the two 60-mL TCK were centrifuged at 1,600×g for 5 min. After the centrifuge, the bottom part of the lipoaspirate was removed. Then, the packed adipose tissue in the TCK was mixed thoroughly with 50 mL of collagenase (collagenase specific for connective tissue (LIBERASE TL) and collagenase specific for adipose tissue (LIBERASE TM)) at a ratio of 1:1 (v:v) by connecting the 60 mL Luer lock syringe via specialized connector. Afterwards, the packed adipose tissue was cut 10-12 times using a homogenizer with blades. The minced lipoaspirate is equally divided into two TCKs, and then two TCK with the mixture are incubated in a rotating incubator mixer at 37° C. for 40 minutes. After the 40 minutes of incubation, they are centrifuged at 800×g to separate and remove collagenase. The supernatant, which contains collagenase, of each TCK was removed and discarded. TCKs were filled with dextrose 5% in the normal saline solution (DSNS) up to 50-55 mL and centrifuged again. This process was repeated for a total of three times. After the last centrifuge, the total volume of the pellet (the SVF containing both ASCs and ECM along with other cells and tissue obtained) was 10 mL. The 10 mL of adipose SVF was isolated on each occasion and then filtered three times using 100 μm metal filters, to remove any large debris. Afterward, the 10 mL filtered adipose SVF was added with 10 mL of normal saline solution. The total of 20 mL allogeneic haploidentical adipose SVF was then injected into the patient as a slow intravenous push (IVP) over 10 minutes. Total, two IVP injections of adipose SVF were performed on two separate occasions, one week apart (FIG. 2).

About 62 days after the second IVP injection of allogeneic, haploidentical adipose SVF, the patient's blood IGF-1 level increased approximately 50% from the level 294.0 to 434.9 ng/mL (FIG. 2). Consequently, 134 days after the second SVF injection, the HGPS patient noted 5 cm growth in height and 1.1 kg of weight gain (FIG. 2). Furthermore, the patient noticed an improved sense of well-being with increased energy, appetite, and food intake. No side effect has been reported.

Other than IGF-1 levels and liver functions test, the available patient's blood results did not show much change from July 2019 to November 2019. The patient has been having chronic elevation of liver function test results, including ALT (alanine aminotransferase) which was reported to be 140 U/l on the prior blood test. For such abnormality, he has been taking the liver vitamins containing ursodeoxycholic acid. The patient discontinued the liver vitamins a few days before the first injection of the adipose SVF. Since the patient previously had abnormally elevated blood liver function test results, it is probable that the discontinuation of the liver vitamins may have caused the elevation of ALT/AST (aspartate aminotransferase) and other liver enzymes, instead of the injections of adipose SVF.

HGPS patients have growth retardation beginning in their childhood due to the laminopathy caused by progerin, which is also shown to accumulate in normal aging human patients (Scaffidi, et al., Science, 312, 1059-1063 (2006)). The average rate of height growth in HGPS patients is about 3.5 cm per year during the ages of 1-10. At the age of around 13, HGPS patients start to have cardiovascular problems, which is comparable to the age of 60s in the general normal population (Sinha, et al., Indian J. Med. Res., 139, 667-674 (2014)). The average age of survival in HGPS patients is approximately 14 years. Thus, the height increase of 5 cm over 4 months in this 13-year-old HGPS patient (FIG. 2) may be equivalent to a growth spurt in a patient in his 60s, which may be considered to be highly unexpected.

Along with the height increase, the patient gained more than 1 kg over the same period (FIG. 2). This weight gain may be significant since the average weight gain in HGPS patients is about 0.5 kg per year. Weights can be variable during certain times of the day depending on oral intakes and bodily secretions. Thus, to ensure the minimal true weight as possible, the weight was measured in the morning, right after waking up and emptying his bladder. Since the other earlier weight measurements were performed after meals in the morning, the chance of the patient weight gain being a daily variation is very unlikely.

Along with the height increase, there was almost a 50% increase in the blood level of IGF-1 (FIG. 2). The steep rise of IGF-1 is highly associated with growth spurt during puberty (Cole, et al., Clin. Endocrinol. (Oxf), 82, 862-869 (2015)). This HGPS patient being 13 years old, having such puberty-like changes during the time of high morbidity rate in the HGPS patient population, may signify that there may be some positive changes at the cellular level. In addition, IGF-1 has been shown to be increased in animal studies to have longevity effects (Marino, et al., Proc. Natl. Acad. Sci. USA., 107, 16268-16273 (2010)). Likewise, the increase in IGF-1 may signify the possibility of extending longevity in this HGPS patient.

It is very well known that mammalian cells naturally release EVs containing various bioactive materials including fragments of DNAs, RNAs, enzymes, proteins, lipids, mitochondria, and other cellular materials and organelles (Gurunathan, et al., Cells, 8, 307 (2019)). Among the three different types of EVs (apoptotic bodies, exosomes, and microvesicles), most data are available with regards to exosomes (Gurunathan, et al., Cells, 8, 307 (2019); Elahi, et al., Stem Cells, 38, 15-21 (2020). After being originated from the endosomal system of each cell, exosomes are released into the extracellular space (Raposo, et al., J. Cell Biol., 200, 373-383 (2013)). While in the extracellular space, exosomes are internalized by host cells via a mechanism involving the fusion of the cell membranes or by phagocytosis. Once internalized, exosomes release their contents into the recipient cells, potentially exerting regenerative or rejuvenating effects by improving or replacing the needed cellular cytoplasmic/nuclear contents (Raposo, et al., J. Cell Biol., 200, 373-383 (2013)). With regards to HPGS, where the correct form of prelamin A is lacking, EVs derived from a healthy individual may transfer normal form of prelamin A with all other materials necessary for the diseased target cells to manufacture the correct form of lamin A.

MSCs are well known to release a large number of exosomes (Elahi, et al., Stem Cells, 38, 15-21 (2020). Further, it is now known that progerin adversely affects functions of adult stem cells in their ability to differentiate and maintain the relevant tissue homeostasis (Scaffidi, et al., Nat. Cell Biol., 10, 452-459 (2008)). Thus, cells from mesoderm-derived tissue such as bone, cartilage, muscles, and vascular epithelium can be more adversely affected in HGPS patients than other tissue. In HGPS patients, when intact donor MSCs are injected intravenously, some of the cells may pass through the pulmonary vasculature and potentially replenish the mesoderm-derived tissues with new functional multipotent stem cells to help maintain the homeostasis (Kallmeyer, et al. Stem Cells Transl. Med., 9, 131-144 (2019); Park, et al., Cell Transplant., 27, 1203-1209 (2018)). This would be another potential mechanism explaining the positive effects shown in this patient.

What is claimed is:

1. A method of treating a subject with Hutchinson-Gilford progeria syndrome, the method comprising administering to the subject a composition comprising:
an allogeneic haploidentical adipose tissue-derived stromal vascular fraction (SVF) produced by a process comprising:
centrifuging an allogeneic haploidentical adipose tissue lipoaspirate to obtain a packed adipose tissue;
mixing the packed adipose tissue with collagenase;
mincing the packed adipose tissue mixed with the collagenase by using a homogenizer;
incubating the minced adipose tissue;
centrifuging the incubated adipose tissue to separate and remove the collagenase; and
repeating the centrifuging to obtain the stromal vascular fraction.

2. The method of claim 1, wherein the administration comprises intravenous push administration.

3. The method of claim 2, wherein the intravenous push administration comprises a first intravenous push administration and a second intravenous push administration one week after the first intravenous push administration.

4. A method of treating a subject with Hutchinson-Gilford progeria syndrome, the method comprising:
preparing allogeneic haploidentical adipose tissue-derived stromal vascular fraction (SVF) from a donor without Hutchinson-Gilford progeria syndrome; and
administering to the subject the allogeneic haploidentical adipose tissue-derived stromal vascular fraction.

5. The method of claim 4, wherein the administration comprises intravenous push administration.

6. The method of claim 5, wherein the intravenous push administration comprises a first intravenous push administration and a second intravenous push administration one week after the first intravenous push administration.
